(19) United States
(12) Patent Application Publication
(10) Pub. No.: US 2013/0216521 A1
(43) Pub. Date: Aug. 22, 2013

(54) DIETARY SUPPLEMENT FOR ENHANCING ANIMAL HEALTH

(71) Applicants: Melinda Fernyhough Culver, Oakland, CA (US); Alison Weber, Bloomfield, NJ (US); Marta Draper, Nyack, NY (US); Tom Myers, Redwood City, CA (US)

(72) Inventors: Melinda Fernyhough Culver, Oakland, CA (US); Alison Weber, Bloomfield, NJ (US); Marta Draper, Nyack, NY (US); Tom Myers, Redwood City, CA (US)

(21) Appl. No.: 13/365,752

(22) Filed: Feb. 3, 2012

(51) Int. Cl. A61K 35/74; A61K 31/702; A61K 31/716; A61K 38/43; A61K 35/20; A61K 39/395; A61K 38/17; A61K 31/202; A61K 36/062

(52) U.S. Cl. CPC A61K 35/741; A61K 31/202; A61K 31/702; A61K 31/716; A61K 35/742; A61K 36/062; A61K 35/747; A61K 35/745; A61K 35/744; A61K 35/20; A61K 39/395; A61K 38/17; A61K 38/43
USPC 424/130.1; 424/93.4; 514/560; 514/54; 424/93.46; 424/93.5; 424/93.45; 424/535; 514/5.5; 424/94.1

(57) ABSTRACT

A dietary supplement system including three general components is provided. The first component is the fatty acid component and this includes sunflower seeds (containing omega 6 fatty acids), flax seed (containing omega 3 fatty acids) and powdered oils (containing omega 3 or omega 6 fatty acids and generally produced from sunflower oils). The weight ratio of omega 6 fatty acids to omega 3 fatty acids is 10:1 to 1:1, with 10:1 to 5:1 being preferred for normal animals and 2:1 to 1:1 being preferred for animals which have fatty acid responsive conditions. The second component is the GI component, which is used for facilitating animal gut health. This component includes prebiotics, probiotics and immunoglobulin (typically from albumin concentrate). The third component is the breed specific component and can include vitamins, minerals, additional fatty acids, antioxidants, amino acids, palatants, and nutraceutical additives.

### BACKGROUND OF THE INVENTION

Diet is important to animal health. A diet should include the proper amount of nutrients, but sometimes this is not possible.

As is well known, certain omega 3 and 6 fatty acids are important to an animal's diet because they cannot be made by most animals and must be supplied in their diet. An omega 6 fatty acid, such as linoleic acid, is considered essential in companion animals because most animals cannot make this material. Fatty acids in the omega 6 family are responsible for growth, reproduction, hormone precursors, and cell membrane and skin health. Omega 3 fatty acids, such as linolenic acid, docosahexaenoic acid (DHA), and eicosapentaenoic acid (EPA), are needed for brain and eye function as well as cell membrane and skin health and function in the anti-inflammatory pathway. Omega 3 fatty acids can also support healthy joints in an animal. By way of example, DHA is needed during periods of early growth for puppies and kittens and for cognition in older animals.

Common cereal grains that are included in dog and cat foods products that are currently available in the marketplace do little to add large amounts of omega 3 fatty acids. Moreover, although omega 6 fatty acids are present in such grain products, the important omega 6 fatty acid (linoleic acid) is present in extremely small amounts. In fact, wheat, corn, and rice, which are typical pet food cereal grains, have little to no naturally occurring linoleic acid, linolenic acid, EPA or DHA. Additionally, the current processes required to manufacture most forms of dog and cat food products, which typically require the use of high heat and/or high sheer, do not support the addition of linoleic acid, linolenic acid, DHA or EPA. This is because large amounts of heat and/or sheer tend to degrade these compounds.

As is also well known, the intestinal tract of companion animals houses up to 70% of the animal's immune system. It is of course important for the overall health of the animal that the intestinal tract and the immune system are healthy and are functioning properly.

Therefore, it would be desirable to provide a dietary supplement for enhancing animal health which incorporated omega fatty acids and which supports the health of the animal's digestive tract.

### SUMMARY OF THE INVENTION

The dietary supplement system of the invention preferably includes three general components. The first required component is the fatty acid component and this includes sunflower seeds (containing omega 6 fatty acids), flax seed (containing omega 3 fatty acids) and perhaps powdered oils (containing omega 3 or omega 6 fatty acids and generally produced from sunflower oils). The weight ratio of omega 6 fatty acids to omega 3 fatty acids is 10:1 to 1:1, with 10:1 to 5:1 being preferred for normal animals and 2:1 to 1:1 being preferred for animals which have fatty acid responsive conditions. The second required component is the GI component, which is used for facilitating animal gut health. This component includes prebiotics, probiotics and immunoglobulin (typically from albumin concentrate). The third component which is optional, but preferred, is the breed specific component and can include vitamins, minerals, additional fatty acids, antioxidants, amino acids, palatants and nutraceutical additives.

### Fatty Acid Component

The inventive system includes omega fatty acids that serve as structural components in cell membranes throughout the body of an animal.

As is well known, omega fatty acids (omega 3, 6, and 9) have double bonds at varying locations along the fatty acid chain.

Of importance to the inventive system is the ratio of fatty acids, specifically the ratio of omega 6 fatty acids to omega 3 fatty acids. In accordance with the invention, the ratio of omega 6 fatty acids to omega 3 fatty acids ranges from 5:1 to 10:1. This ratio is reduced to from 2:1 to 1:1 for animals with a fatty acid responsive condition such as atopy, arthritis, and dermatitis. In contrast, current pet food products can have a fatty acid ratio of omega 6 fatty acids to omega 3 fatty acids from 20:1 to 40:1.

### GI Component

In accordance with the invention, several ingredients are included for the purpose of increasing the function of both the intestinal tract and the associated lymphoid tissue.

One such ingredient is referred to as prebiotics, which are different groups of fibers that are normally not digested by the animal. Prebiotics can support the growth of beneficial gut bacteria, but do not support the growth of unhealthy gut microbes. There are many types of prebiotic fibers, but the most common ones are MOS (mannanoligosaccharides), FOS (fructooligosaccharides), and inulin.

Another such ingredient is referred to as probiotics, which are live bacterial cells that impart beneficial effects on the animal when ingested. Probiotics promote intestinal health, help prevent gastrointestinal disease and diarrhea, support a healthy immune system, and maintain a healthy digestive system in times of stress, traveling, or boarding.

In accordance with the invention, prebiotics provide a nutrient supply for probiotics to thrive. The combination of a mixture of probiotics and prebiotics is thus “symbiotic”.

A third such ingredient is IgG (immunoglobulin), which provides non-specific immunity for companion animals against common GI toxins, harmful bacteria, and viruses. Additionally, the addition of IgG to the diet of young animals can decrease the clinical signs of disease, shorten the course of intestinal diseases, and increase weight gain and growth. Lastly, the addition of IgG in the diet provides for immunomodulation in that it can stimulate the animal's own immune system.

Accordingly, it is an object of the invention to provide an improved dietary supplement which enhances animal gut health.

Another object of the invention is to provide for an animal dietary supplement that provides nutritional balance.

Still a further object of the invention is to provide for an animal dietary supplement which facilitates improved intake and adsorption of nutrients.

Other objects and advantages of the invention will in part be obvious and in part will be apparent from the description herein.

### DETAILED DESCRIPTION OF THE INVENTION

The omega fatty acid component active ingredients are present in the inventive formulation in an amount between about 13 and 22 weight percent; it consists of a blend of omega 6 fatty acids and an omega 3 fatty acids in a weight ratio of between about 1:1 and 10:1. The preferred omega 6 fatty acid is linoleic acid, although other biologically important fatty acids may be used such as arachadonic acid and γ-linolenic acid. The preferred omega 3 fatty acid is linolenic acid; others include eicosapentaenoic acid and docosahexaenoic acid. The omega 6 fatty acids are present in the inventive formulation from between about 6 and 10 weight percent and are present in the omega fatty acid component from between about 8 and 12 weight percent. The omega 3 fatty acids are present in the inventive formulation from between about 5 and 10 weight percent and are present in the omega fatty acid component from between about 7 and 11 weight percent.

Linoleic acid (the preferred omega 6 fatty acid) is considered, for purposes of the inventive system, an essential fatty acid in that it cannot be synthesized, de novo, by the body and, therefore, must be supplied in the diet. Arachadonic acid is also considered to be an important additional fatty acid for cats as this species has a virtually no ability to synthesize this particular fatty acid. Likewise, omega 3 fatty acids cannot be synthesized by companion animals. However, animals have a limited ability to form eicosapentaenoic acid and docosahexaenoic acid from \acute{α}-linolenic acid. This is because they lack the appropriate fatty acid enzymes.

The preferred source for linoleic acid is sunflower seed. Other sources of linoleic acid may include, but are not limited to, rapeseed, corn, cottonseed, blackcurrant seed, soybeans, and safflower.

The preferred source of linolenic acid is flax seed (brown or golden varieties); it can also be sourced from chia seed, lignonberry, and hempseed. The omega 3 fatty acids eicosapentaenoic acid and docosahexaenoic acid are found only in marine sources. The preferred sources of these two are marine algae and fish oil or fish meal.

In the preferred form of the invention, the two fatty acids are linoleic acid and linolenic acid in a weight ratio of between about 1:1 and 10:1.

In accordance with the inventive formulation, an optional source for either the omega 3 or omega 6 fatty acids is from a dried or powdered oil. This optional source can partially be substituted for either the linoleic acid or linolenic acid ingredient that is obtained from the sources identified hereinabove. Omega 3 and/or omega 6 fatty acids from dried or powdered oils are advantageous in packaging this formulation in certain highly desired packaging formats. The preferred dried oil or powdered oil is either flaxseed oil or sunflower seed oil although other oils such as fish oil, linseed oil, and borage oil, for example, may be used. It is preferred that the oil be dried on a base of rice or tapioca.

The preferred weight ratio of linoleic acid to \acute{α}-linolenic acid is 10:1 to 1:1, as set forth hereinbefore, with 10:1 to 5:1 being preferred for normal animals and 2:1 to 1:1 being preferred for animals which have fatty acid responsive conditions. Such conditions are common to the animal world and may include, but are not limited to, atopy, dry skin, lackluster haircoat, arthritis, and dementia. In contrast, most available commercial food products for companion animals have a ratio of linoleic acid to \acute{α}-linolenic acid of between about 20:1 and 40:1.

In a preferred embodiment of the overall inventive formulation, flaxseed, which contains from between about 18 and 24 weight percent linoleic acid, is added at approximately 28-38 weight percent, sunflower seed, which contains between about 22 and 25 weight percent linolenic acid, is added at approximately 28-38 weight percent (equal to flaxseed), and powdered sunflower oil, which contains between about 3 and 6 weight percent linolenic acid is added at approximately 7-11 weight percent.

In this preferred embodiment, and in terms of the omega fatty acid component itself, flaxseed is added at approximately 40-45 weight percent, sunflower seed is added at approximately 40-45 weight percent, and powdered sunflower oil is added at approximately 10-20 weight percent.

It is noted that sunflower seeds contain far greater amounts of omega 6 fatty acids than the omega 3 fatty acids that are contained in flaxseeds. Therefore, in the preferred embodiment herein discussed, a ratio of linoleic acid to \acute{α}-linolenic acid at approximately 2:1 is provided.

Omega Component

| | Percent in | Percent in |
|---|---|---|
| Ingredient | component | final formula |
| Omega 3 oilseed | 40-45% | 28-38% |
| Omega 6 oilseed | 40-45% | 28-38% |
| Omega 3 or 6 powdered oil | 10-20% | 7-11% |

It should be noted that for each ingredient in the omega fatty acid component, there is a preferred form. For example, flax seed in its native form is covered in a tough outer coating that protects the fatty acids from oxidation and degradation. However, this coating also makes the fatty acids somewhat resistant to digestion as the coating first needs to be broken. In animals without a large grinding component to mastication (e.g. carnivores and omnivores), this is nearly impossible. Therefore, it is necessary for the seed coating to first be opened for the seed to be nutritional for the animal. This can be accomplished by soaking (softens the outer coating), by grinding the seed (generating producing flax seed meal), or by slicing each of the seeds into a smaller particle size. The preferred form is sliced as this form can more easily be handled by conventional packaging machinery than the less preferred meal form. Since less processing is done to the seed and the particle size of the seed is greater, less of the fatty acids are released as free oil, making this form less likely to form an obstruction in the packaging machine. Care should of course be taken when slicing the flax seed so that minimal to no frictional heat is introduced from the slicing machine that is used to perform the processing (so-call “cold processed”, a proprietary process of Heartland Flax of North Dakota); this preserves the integrity of the omega fatty acids within the seed. This above mentioned processing of flax seed holds true for the sunflower seed as well, although this seed usually has already been separated from its husk. It is important that the sunflower seed not be ground into a meal as this seed tends to have high oil content and will freely release the oil, resulting in significant problems during packaging.

To offset the level of free oil, the last (though optional) ingredient in the omega fatty acid component, namely the powdered or dried oil, is introduced in order to keep the level of omega fatty acid within the desired ratio, but allows for greater flow during packaging.

The GI component active ingredients are preferably present in the inventive formulation in an amount between about 1 and 5 weight percent; they include prebiotics, probiotics, and immunoglobulin. Prebiotics are preferably present in the inventive formulation from between about 1.0 and 4.0 percent by weight and are preferably present in the GI component from between about 31 and 38 percent by weight. Prebiotics are groups of fibers that are not digested by the animal and which can support the growth of beneficial GI bacteria, but which do not support the growth of unhealthy gut microbes. There are many types of prebiotic fibers with the most preferred being man nanoligosaccharides. Other suitable prebiotic fibers include fructooligosaccharides, xylooligosaccarides, galactooligosaccarides, inulins, and β-glucans. Prebiotics may be obtained from yeast, chicory root, and beet pulp. The preferred source is from yeast as the wall of yeast contains both mannanoligosaccharides and β-glucans. In the preferred formulation of the invention, yeast, which contains from between about 50 and 55 percent prebiotic fibers, is preferably added to the inventive formulation at approximately 3-7 weight percent, and to the GI component at approximately 63-68 weight percent.

Probiotics are preferably present in the inventive formulation in an amount between about 0.01 and 0.1 percent by weight and are preferably present in the GI component from between about 0.1 and 1.0 percent by weight. Probiotics are live bacterial cells that impart beneficial effects on an animal when they are ingested. While there are numerous types of probiotics, there are certain ones which are preferred in the inventive system. The preferred inventive formulation will contain between 3 to 6 different species of probiotics. The species are selected from the group consisting of Aspergillus spp., Bacillus spp., Bacteroides spp., Bifidobacterium spp., Lactobacillus spp., Pediococcus spp. and Enterococcus spp. Probiotics are added as a dried form of the microbes and should be added in an amount such that the animal will receive a minimum of 250,000 CFU/day to a maximum of 1 Billion CFU/day. In a preferred form of the invention, the dried microbes which contain 100% probiotics, are added at approximately 0.1-1 weight percent in the GI component and 0.01-0.1 weight percent in the formulation.

By way of one example, suitable probiotics may contain an equal mix of Bifidobacterium animalis, Enterococcus diacetylactis, Lactobacillus acidophilus, Lactobacillus casei and Lactobacillus lactis. These probiotics are added to the inventive formulation in order to provide at least 250,000 CFU/day to the animal. The actual inclusion rate of the probiotic mixture will be dependant on the concentration of bacteria in the ingredient (CFU/g) and the feeding instructions of the final product. In one preferred embodiment, the probiotic was included so that every teaspoon (3 g) of the final product contains 250,000 CFU.

The third optional, though preferred, ingredient of the GI component is the animal protein immunoglobulin (Ig). If included, it is preferably present in the inventive formulation in an amount up to about 2 weight percent and in the inventive formulation and in an amount up to 6 weight percent in the GI component. Immunoglobulins are endogenous proteins that provide an animal with protection from bacteria, viruses, and toxins. The use of IgG provides for non-specific immunity against common GI toxins, harmful bacteria, and viruses. Additionally, the addition of IgG to the diet of young animals can decrease the clinical signs of diseases, shorten the course of intestinal diseases, and increase weight gain and growth. Lastly, the addition of IgG in the diet provides for immunomodulation in that it can stimulate the animal's own immune system.

There are 5 isotypes of immunoglobulins: IgG, IgA, IgE, IgM, and IgD, each with its own biological relevance to the animal. IgG is the preferred immunoglobulin, but any of the other identified immunoglobulins may be used. IgG is most commonly found intravascularly and is in high concentrations in blood products. The preferred source of IgG is albumin, but IgG may also be obtained from other blood fractions such as plasma, whole blood or serum, as well as colostrum, milk peptides and enzymes. The preferred animal source for the albumin is cow, but is not limited to this species as pig or another animal may be used. In the preferred formulation, albumin which typically contains from between about 10 and 16 weight percent IgG, is added in an amount up to 10 weight percent in the overall formulation and up to 37 weight percent in the GI component.

Additionally, the amount of desired Ig may adjusted depending upon the specific animal (young animals require greater GI protection as their innate immune system is still naïve; aged animals require greater GI protection because immune system function decreases with age).

GI Component

| | Percent in | Percent in |
|---|---|---|
| Ingredient | component | final formula |
| Yeast | 63-68% | 3-7% |
| Probiotic microbes | 0.1-1.0% | 0.01-0.1% |
| Albumin concentrate | 32-37% | 0-10% |

In terms of the GI component, it is desirable to have yeast added at approximately 63-68 weight percent, probiotic microbes added at approximately 0.1-1 weight percent, and albumin added at approximately 32-37 weight percent.

The one or more compounds of the optional breed specific component may be present in the inventive formulation in an amount between about 10 and 17 weight percent. Such compounds include several ingredients that are potentially beneficial to a specific animal breed or groups of animal breeds. Indeed, there may be as many inventive formulations as there are breeds of dogs and cats. However, in the preferred inventive embodiment, several breeds of animals are grouped together based on similar ailments, thereby requiring similar nutritional support. For example, several different animal breeds are classified into three different breed groups: toy breeds, active breeds, and medium/large breeds. The toy grouping includes Maltese, Shih Tsu, Lhasa Apso, and Yorkshire Terrier dogs. The active breed grouping includes all terriers, Pugs, and French Bulldogs. The medium/large breed grouping includes retrievers, spaniels, setters, Bulldogs, Boxers, and German Shepherd Dogs.

The inventive formulation that is most suitable for each of these groupings is set forth below. That said, other suitable ingredients may be substituted.

The breed specific component shall be added to the omega fatty acid component and the GI component at the appropriate levels in order to obtain a final blend of ingredients.

### Example 1 of Breed Specific Component

### Formulas

Active Small Breed

| | % of | % of |
|---|---|---|
| Ingredient | component | formula |
| Palatant | 7-14% | 0.5-3.5% |
| Flow agent | 5-8% | 0.5-3% |
| DHA | 15-25% | 4-6% |
| Egg- Shell Membrane | 4-10% | 0.5-3% |
| Taurine | 17-25% | 3-7% |
| L-carnitine | 31-39% | 6-10% |

### Example 2 of Breed Specific Component

### Formulas

Toy Small Breed

| | % of | % of |
|---|---|---|
| Ingredient | component | formula |
| Palatant | 13-19% | 0.5-3.5% |
| Flow agent | 10-14% | 0.5-3% |
| DHA | 48-53% | 6-9% |
| Vitamin E | 8-11% | 0.5-3% |
| Vitamin C | 4-7% | 0.1-1.5% |
| Zinc Amino Acid Chelate | 0.5-3% | 0.1-1% |
| Biotin | 0.5-3% | 0.1-1% |
| Beta Carotene | 0.5-3% | 0.05-1% |
| Vitamin C | 0.5-3% | 0.1-1% |
| Vitamin A | 0.01-1% | 0.01-1% |

### Example 3 of Breed Specific Component

### Formulas

Medium/Large Breed

| | % in | % in |
|---|---|---|
| Ingredient | component | formula |
| Palatant | 17-20% | 0.5-3.5% |
| Flow agent | 13-16% | 0.5-3% |
| DHA | 7-10% | 0.5-1.5% |
| Taurine | 11-14% | 0.5-3% |
| L-carnitine | 25-28% | 1-5% |
| Vitamin E | 7-10% | 0.1-2% |
| Vitamin C | 7-10% | 0.5-1.5% |
| Vitamin A | 0.1-1% | 0.01-1% |
| Vitamin B₂ Riboflavin | 0.1-1% | 0.01-1% |

This formulation may take the form of lifestage specific for different ages of animals. For example, a canine formula may be broken down into three different stages—puppy (for dogs up to 1 year), adult (for small breed dogs aged 1-7 years or large breed dogs from 1-6 years), and senior (for small breed dogs >7 years or large breed dogs >6 years).

Feline formulations may have additional categories as cats generally have an increased life span over that of dogs. The life stages in cats may be represented, preferably, as kitten (0-12 months), adult (1-6 years), senior (7-10 years), and geriatric (>11 years). However, this scheme may be truncated to kitten (0-12 months), adult (1-6 years), and senior (>7 years). The following are examples of lifestage specific formulas, although others may be possible.

### Example 1 of Lifestage Specific Component

### Formulas

Senior dog blend

| | % in | % in |
|---|---|---|
| Ingredient | component | formula |
| Palatant | 15-20% | 0.5-3.5% |
| Flow agent | 10-15% | 0.5-3% |
| L-carnitine | 15-20% | 1-5% |
| DHA | 3-6% | 0.1-3% |
| Taurine | 7-10% | 0.1-3% |
| Vitamin E | 2-5% | 0.1-1.5% |
| Vitamin C | 2-5% | 0.1-1.5% |
| Vitamin A | 0.01-1% | 0.01-1% |
| Riboflavin | 0.1-1% | 0.01-1% |
| Glucosamine Sulfate | 27-32% | 2-5% |

### Example 2 of Lifestage Specific Component

### Formulas

Puppy blend

| | % in | % in |
|---|---|---|
| Ingredient | component | formula |
| Palatant | 15-20% | 0.5-3.5% |
| Flow agent | 11-15% | 0.5-3% |
| DHA | 20-24% | 2-6% |
| Taurine | 25-30% | 3-7% |
| Vitamin E | 10-14% | 0.5-3% |
| Vitamin C | 5-9% | 0.1-1.5% |
| Vitamin A | 0.1-1.5% | 0.01-1% |
| Riboflavin | 0.1-1.5% | 0.01-1% |
| Folic Acid | 0.01-1% | 0.01-1% |
| Vitamin B₁₂ | 0.0001-0.001% | 0.0001-0.01% |

### Example 3 of Lifestage Specific Component

### Formulas

Senior cat blend

| | % in | % in |
|---|---|---|
| Ingredient | component | formula |
| Flow agent | 15-19% | 0.5-3% |
| Egg- Shell Membrane | 13-17% | 0.5-3% |
| Vitamin E | 13-16% | 0.1-2% |
| Vitamin C | 4-7% | 0.1-1.5% |
| Beta Carotene | 0.5-1.5% | 0.01-0.5% |
| Vitamin A | 0.1-1% | 0.01-1% |
| L-Lysine | 45-50% | 4-8% |

### Example 4 of Species Specific Component

### Formulas

Adult cat blend

| | % in | % in |
|---|---|---|
| Ingredient | component | formula |
| Flow agent | 10-14% | 0.5-3% |
| L-carnitine | 52-55% | 7-10% |
| Taurine | 26-29% | 3.5-6% |
| Vitamin E | 3-5% | 0.1-2% |
| Vitamin C | 2.5-4% | 0.1-1.5% |
| Vitamin A | 0.1-3% | 0.01-1% |
| Riboflavin | 0.1-3% | 0.01-1% |
| Vitamin B₁₂ | 0.01-1% | 0.0001-0.01% |

Despite the physiologic similarities between dog breeds, each breed is subject to different types of maladies. Additionally, there are many variables within different breeds of dogs, including body size, haircoat, lifestyle, and longevity. When reviewing these variables across dog breeds, it is possible to group these breeds based on these variables. This in turn has allowed for the development of inventive formulations that are better able to address these variables. As such, each formulation has specific ingredients in them for addressing the particular variable at issue. The formulations outlined here are intended to be examples of the invention and this discourse is not intended to be an entire list.

One formulation has been developed to address the needs of toy breed dogs such as the Maltese, Yorkshire Terrier, Lhasa Apso, Shih Tzu, and the Japanese Chin. These breeds have an increased need for skin and coat support for their continuously growing haircoat. Therefore, suitable inventive formulations preferably contain biotin, zinc, and DHA, all of which support healthy skin, a shiny coat, and increased haircoat demand.

For toy breed dogs, other ingredients are possible for the inventive formulation. These breeds are known for a long life span; the inventive formulation may also include antioxidants to support the immune system and overall good health of the animal. Some examples of suitable antioxidants include vitamin C, vitamin E, polyphenols, bioflavinoids, carotenoids, selenium, grape seed extract, milk thistle, \acute{α}-lipoic acid, ubiquinone, phytosterols, and green tea extract.

Toy breed dogs are also prone to increased dental disease. As a result, vitamin C may be added to the inventive formulation for increasing dental support. Other ingredients that may be added for dental support include sodium hexametaphosphate or some other phosphate group, Fucans (brown algae), natural abrasive, or malic acid, zinc citrate, \acute{α}-lipoic acid, oral specific probiotics, or esterified fatty acid complex.

Another grouping of dog breeds includes the West Highland White Terrier, Pug, Scottish Terrier, French Bulldog, Schipperke, Jack Russell Terrier, Parson Russell Terrier, and the Cairn Terrier. These breeds are hardy and active and, therefore, require additional nutritional support for promoting joint health. These breeds have a predisposition towards atopy/allergies and weight gain (despite their high activity). The formula in accordance with the invention is designed to address these issues and includes adding DHA (for atopy/allergies), egg shell membrane (for joint support), and taurine and carnitine (for lean muscle mass and cardiac support). Other possible ingredients which may be added to the inventive formulation include eicosapentaenoic acid (for atopy/allergies); chondroitin, hyaluronic acid, methylsulfonylmethane, glucosamine, esterified fatty acid complex, Chinese Skullcap, and Black Catechu (for joint support); and creatine and ubidiquinone (for lean muscle support).

A third grouping of dog breeds includes Boxers, Brittany Spaniels, Cocker Spaniels, Cavalier King Charles Spaniels, Bulldogs, Labrador Retrievers, Golden Retrievers, Springer Spaniels, and German Shepherd Dogs. These breeds are grouped together because of their tendency towards atopy/allergies, eye disease, heart disease, and weight gain. Therefore, it is preferred that the inventive formulation include DHA (for atopy/allergies); carnitine (for lean muscle mass), taurine (for cardiac support), and vitamin A, vitamin E, vitamin B₂ and taurine (for eye health and vision support). Other ingredients that may be added include eicosapentaenoic acid (for atopy/allergies); creatine and ubidiquinone (for lean muscle support); ubidiquinone, polyphenols, bioflavinoids (for cardiac support), and bilberry and carotenoids such as astaxanthan, carotene, and lycopene (for vision and eye support).

Other formulas made in accordance with the invention are designed to address specific stages of a dog or cat's life (e.g. puppy, senior). For example, a formula designed for a young animal (puppy or kitten) preferably contains ingredients that are beneficial to a growing animal and may well address factors such as the developing heart, muscles, brain, and eyes of the animal. Such a formula may include, but is not limited to, DHA, taurine, and immune support ingredients such as antioxidants, vitamin E, vitamin C, vitamin A, and B vitamins.

A formula made in accordance with the invention that is specific to senior animals will preferably address issues such as reduced movement, a fading immune system, maldigestion and an increased nutritional plane, less retention of lean body mass, age induced sarcopenia, fading vision, cognition and battle age-related senility, and heart disease. Ingredients to include in such a formula include DHA (for cognition); carnitine (for lean muscle mass), taurine (for cardiac support), vitamin A, vitamin E, vitamin B₂ and taurine (for eye health and vision support), and egg shell membrane and glucosamine (for joint support).

This is not intended to be a limiting list of ingredients to be included in an inventive formulation that is designed for senior animals, and other ingredients are possible such as: medium chain triglycerides, lecithin, and phospholipids (for cognition), creatine and ubidiquinone (for lean muscle support); ubidiquinone, polyphenols, bioflavinoids (for cardiac support), bilberry and carotenoids such as astaxanthan, carotene, and lycopene (for vision and eye support), chondroitin, hyaluronic acid, methylsulfonylmethane, glucosamine, esterified fatty acid complex, and Chinese Skullcap, and Black Catechu (for joint support).

Lastly, as cats are not the same as small dogs, certain formulations made in accordance with the invention are designed to address the specific needs of the feline population. As compared to formulations that are designed for dogs, a formulation that is specific for cats includes the addition of lysine. The amino acid lysine has been shown to have some efficacy in reducing the clinical signs and decreasing shedding of several strains of herpes viruses. Although numerous species of herpes viruses exist, each will cause different disease states in their host species. The feline herpes virus is a ubiquitous virus transmitted through aerosol and exposure occurs in early kittenhood. This virus causes upper respiratory (sneezing, coughing, nasal discharge, ocular ulceration) difficulties in cats and can be severe enough to even in its attack as to cause death in the very young. Additionally, a feline herpes virus condition is almost always never completely abated and may therefore act out in times of illness or stress to the cat. For this reason, an inventive formula for cats should contain lysine.

Additionally, cats are unable to synthesize the amino acid taurine on their own so it is most preferred that they receive taurine as part of their ever-day diet; it is well known that deficiencies in this amino acid have been linked to heart disease. For this reason, the inventive formulation as applied cats includes additional taurine.

Cats are susceptible to obesity induced diabetes (similar to human but dissimilar to dogs). Couple this with the need for a cat (an obligate carnivore) to consume high protein and fat in the diet as compared to a dog (an omnivore) can lead an obese cat to be lacking essential fatty acids. Additionally, long life spans are plentiful among the cat population with many reaching >15 years.

Antioxidants are essential to protecting the body's cells against oxidative stress. Many conditions are similar between cats and dogs; however, cats may be more sensitive to an ingredient and require a different dosage or a substitution. A suitable feline formula made in accordance with the invention may include taurine and/or carnitine for cardiac support, canitine for promoting lean body mass, one or more joint/mobility support ingredients such as egg shell membrane, chondroitin, hyaluronic acid, methylsulfonylmethane, glucosamine and esterified fatty acid complex), lysine for promoting upper respiratory health, one or more antioxidants such as vitamin C, vitamin E, polyphenols, bioflavinoids, carotenoids, selenium, grape seed extract, milk thistle, \acute{α}-lipoic acid, ubiquinone, phytosterols, and green tea extract, and one or more cognitive support ingredient such as DHA, medium chain triglicerides, lecithin, phospholipids.

The preferred form of the inventive formulation is a powder or meal that is top dressed onto the animal's diet (dry or wet). However, other forms should not be excluded such as granular, pelleted, or crumbled form that is top dressed onto the animal's diet (dry or wet). Additionally, if possible, any of these forms may be mixed in to the animal's diet or be given directly to the animal (i.e. not mixed or top dressed to the diet). Any form of the inventive formulation may be packaged in larger bulk bags (8-16 oz) or, if preferred, into single serve packets or stick packs.

In the preferred method for preparing the inventive product, compounds from the fatty acid component are first mixed with the compounds from the GI component until uniform. Then, the compounds of the optional breed specific component are added and mixed until uniform. Modifications may be made in processing depending on the actual machine used for mixing and the procedure that is needed for filling the mixture into its packaging. Additionally, the exact number and actual ingredients used is dependent on which formula is to be made.

The below example is but one possible method and others are possible. Selected ingredients are added into an industrial blender of an appropriate size for the desired batch and which will allow for proper blend uniformity. The following initial ingredients are added in the described order by first weighing out all the ingredients and placing each into a paper bag or stainless steel bowl. These shall be called Group 1 ingredients. Each Group I ingredient is added to the blender and blended on “high” for one minute. To ensure that proper mixing has occurred, one checks for color uniformity. If proper color uniformity has not been achieved, the mixer may be blended on low for an additional one minute. It should be noted that blending at high speeds and/or blending for extended periods of time should be avoided in order to maintain fatty acid integrity as well as to keep the product from becoming too oily as this will retard its flow through the packaging machinery.

Group 1

| Order | Ingredient |
|---|---|
| 1 | Omega 6 oilseed |
| 2 | Omega 3 oilseed |
| 3 | Oil Powder |
| 4 | Albumin concentrate |
| 5 | Prebiotic |
| 6 | Palatant |
| 7 | Flow agent |
| 8 | Probiotic microbes |

To the above Group 1 mixture, one adds the following Group 2 ingredients (they should have been weighed and placed into a paper bag or stainless steel bowl). The Group II ingredients are blended for 4 minutes on high speed. Proper mixing will have occurred when color is uniform; if color uniformity is not achieved then blend the mixture on low for an additional 1 minute. Again, blending at high speeds and/or blending for extended periods of time should be avoided in order to maintain fatty acid integrity as well as to keep the product from becoming too oily as this will retard its flow during packaging.

Group 2

| Order | Ingredient |
|---|---|
| 9 | DHA |
| 10 | Vitamin E Acetate 50% |
| 11 | Vitamin C |
| 12 | Zinc |
| 13 | Biotin |
| 14 | Beta Carotene |
| 15 | Vitamin A |

The following is a flow diagram outlining the steps to blending this product on the equipment used in this embodiment.

The following are specific examples of the inventive formulation as prepared from the preferred source ingredients.

### Example Formula #1

| | |
|---|---|
| omega 6 oilseed (sunflower seed) | 33.173% |
| omega 3 oilseed (flax seed) | 33.173% |
| dried oil (sunflower) | 8.503% |
| albumin concentrate | 7.655% |
| L-lysine | 5.000% |
| prebiotic (yeast) | 4.256% |
| palatant (yogurt) | 2.552% |
| flow agent (rice hulls) | 1.823% |
| egg shell membrane | 1.667% |
| vitamin E | 1.515% |
| vitamin C | 0.533% |
| beta carotene | 0.107% |
| vitamin A | 0.029% |
| probiotic microbes | 0.014% |
| Total | 100.000% |

### Example Formula #2

| | |
|---|---|
| omega 6 oilseed (sunflower seed) | 31.406% |
| omega 3 oilseed (flax seed) | 31.406% |
| L-carnitine | 8.333% |
| dried oil (sunflower) | 8.050% |
| albumin concentrate | 7.248% |
| taurine | 4.167% |
| prebiotic (yeast) | 4.029% |
| palatant (yogurt) | 2.416% |
| flow agent (rice hulls) | 1.726% |
| vitamin E | 0.590% |
| vitamin C | 0.533% |
| vitamin B₂ | 0.050% |
| vitamin A | 0.029% |
| probiotic microbes | 0.013% |
| vitamin B₁₂ | 0.003% |
| Total | 100.000% |

### Example Formula #3

| | |
|---|---|
| omega 6 oilseed (sunflower seed) | 32.606% |
| omega 3 oilseed (flax seed) | 32.606% |
| dried oil (sunflower) | 8.358% |
| albumin concentrate | 7.524% |
| prebiotic (yeast) | 4.183% |
| taurine | 4.167% |
| docosahexanioc acid | 3.333% |
| palatant (yogurt) | 2.509% |
| flow agent (rice hulls) | 1.792% |
| vitamin E | 1.788% |
| vitamin C | 1.000% |
| vitamin B₂ | 0.080% |
| vitamin A | 0.028% |
| probiotic microbes | 0.013% |
| folic acid | 0.010% |
| vitamin B₁₂ | 0.003% |
| Total | 100.000% |

### Example Formula #4

| | |
|---|---|
| omega 6 oilseed (sunflower seed) | 32.846% |
| omega 3 oilseed (flax seed) | 32.846% |
| dried oil (sunflower) | 8.419% |
| albumin concentrate | 7.580% |
| prebiotic (yeast) | 4.214% |
| glucosamine | 4.000% |
| palatant (yogurt) | 2.527% |
| L-carnitine | 2.500% |
| flow agent (rice hulls) | 1.805% |
| taurine | 1.250% |
| docosahexanioc acid | 0.750% |
| vitamin C | 0.670% |
| vitamin E | 0.500% |
| vitamin B₂ | 0.057% |
| vitamin A | 0.022% |
| probiotic microbes | 0.014% |
| Total | 100.000% |

### Example Formula #5

| | |
|---|---|
| omega 6 oilseed (sunflower seed) | 33.250% |
| omega 3 oilseed (flax seed) | 33.250% |
| dried oil (sunflower) | 8.523% |
| albumin concentrate | 7.673% |
| prebiotic (yeast) | 4.266% |
| palatant (yogurt) | 2.558% |
| L-carnitine | 3.571% |
| flow agent (rice hulls) | 1.827% |
| taurine | 1.786% |
| vitamin E | 1.143% |
| docosahexanioc acid | 1.070% |
| vitamin C | 0.957% |
| vitamin B₂ | 0.081% |
| vitamin A | 0.031% |
| probiotic microbes | 0.014% |
| Total | 100.000% |

### Example Formula #6

| | |
|---|---|
| omega 6 oilseed (sunflower seed) | 32.115% |
| omega 3 oilseed (flax seed) | 32.115% |
| dried oil (sunflower) | 8.232% |
| docosahexanioc acid | 8.333% |
| albumin concentrate | 7.411% |
| prebiotic (yeast) | 4.120% |
| palatant (yogurt) | 2.470% |
| flow agent (rice hulls) | 1.765% |
| vitamin E | 1.637% |
| vitamin C | 1.060% |
| zinc amino acid chelate | 0.303% |
| biotin | 0.213% |
| beta carotene | 0.183% |
| vitamin A | 0.030% |
| probiotic miccrobes | 0.013% |
| Total | 100.000% |

### Example Formula #7

| | |
|---|---|
| omega 6 oilseed (sunflower seed) | 29.601% |
| omega 3 oilseed (flax seed) | 29.601% |
| L-carnitine | 8.333% |
| dried oil (sunflower) | 7.587% |
| albumin concentrate | 6.831% |
| docosahexanioc acid | 4.500% |
| taurine | 4.167% |
| prebiotic (yeast) | 3.798% |
| palatant (yogurt) | 2.277% |
| egg shell membrane | 1.667% |
| flow agent (rice hulls) | 1.626% |
| probiotic microbes | 0.012% |
| Total | 100.000% |

The inventive system has the following advantages:
it preferably uses high quality oil seeds that are chopped (not ground) in a cold process in order to minimize damage to seed structure, thereby minimizing free oil available for oxidation;
it uses a proprietary cold slice processing system;
it prevents heat damage to any oil that may have been liberated from the seeds, thus minimizing oxidation and other degradative reactions;
it optimizes the omega 3 and 6 fatty acid ratio;
it provides a balanced approach which meets or exceeds the amount of nutrients that are typically missing in companion animal retail foods;
it provides for GI health in animals, thereby enhancing the uptake of the provided nutrients; and
it adds ingredients not normally find in typical animal food sources and targets only what a specific animal breed or specific lifestage of an animal may be lacking in nutritional support.

The scope of the invention will now be set forth in the following claims.

1. A dietary supplement formulation comprising;
a fatty acid component comprising an omega 6 fatty acid and an omega 3 fatty acid; and
a gastrointestinal component comprising prebiotics and probiotics.

2. The dietary supplement formulation of claim 1, further including a breed specific component selected from the group consisting of vitamins, minerals, additional fatty acids, antioxidants, amino acids, palatants and nutraceutical additives.

3. The dietary supplement formulation of claim 1, wherein the omega 6 fatty acid is selected from the group consisting of linoleic acid, arachadonic and γ-linolenic acid.

4. The dietary supplement formulation of claim 1, wherein the omega 3 fatty acid is selected from the group consisting of linolenic acid, docosahexaenoic acid (DHA) and eicosapentaenoic acid (EPA).

5. The dietary supplement formulation of claim 1, wherein the omega 6 fatty acid is linoleic acid and wherein the linoleic acid is sourced from the group consisting of sunflower seeds, rapeseed, corn, cottonseed, blackcurrant seed, soybeans and safflower.

6. The dietary supplement formulation of claim 1, wherein the omega 3 fatty acid is linolenic acid and wherein the linolenic acid is sourced from the group consisting of flax seed, chia seed, lignonberry, and hempseed.

7. The dietary supplement formulation of claim 4, wherein docosahexaenoic acid (DHA) and eicosapentaenoic acid (EPA) are sourced from the group consisting of marine algae, fish oil and fish meal.

8. The dietary supplement formulation of claim 1, wherein the weight ratio of omega 6 fatty acids to omega 3 fatty acids is from between about 10:1 to 1:1.

9. The dietary supplement formulation of claim 1, wherein the fatty acid component further includes one or more dried or powdered oils.

10. The dietary supplement formulation of claim 9, wherein said one or more dried or powdered oils are selected from the group consisting of flaxseed oil, sunflower oil, fish oil, linseed oil and borage oil.

11. The dietary supplement formulation of claim 9, wherein the omega 6 fatty acid is sourced from sunflower seed present in the formulation in an amount between about 25 and 38 weight percent; the omega 3 fatty acid is sourced from flaxseed present in the formulation in an amount between about 28 and 38, and the one or more dried or powdered oils is sunflower oil present in the formulation in an amount between about 7 and 11 weight percent, the weight percents based on the total weight of the formulation.

12. The dietary supplement formulation of claim 11, wherein the flaxseed has a seed coating that is opened by one of soaking, grinding and slicing.

13. The dietary supplement formulation of claim 1, wherein the prebiotics are selected from the group consisting of mannanoligosaccharides, fructooligosaccharides, xylooligosaccarides, galactooligosaccarides, inulins, and β-glucans.

14. The dietary supplement formulation of claim 1, wherein said prebiotics are obtained from the group consisting of yeasts, chicory root and beet pulp.

15. The dietary supplement formulation of claim 1, wherein the probiotics are selected from the group consisting of Aspergillus spp., Bacillus spp., Bacteroides spp., Bifidobacterium spp., Lactobacillus spp., Pediococcus spp. and Enterococcus spp.

16. The dietary supplement formulation of claim 1, wherein said gastrointestinal component further includes an animal protein selected from the group consisting of immunoglobulin, colostrum, whole blood, milk peptides and enzymes.

17. The dietary supplement formulation of claim 16, wherein the animal protein is immunoglobulin selected from the group of isotypes consisting of IgG, IgA, IgE, IgM and IgD.

18. The dietary supplement formulation of claim 2, wherein the breed specific component includes an antioxidant selected from the group consisting of vitamin C, vitamin E, polyphenols, bioflavinoids, carotenoids, selenium, grape seed extract, milk thistle, \acute{α}-lipoic acid, ubiquinone, phytosterols and green tea extract.

19. The dietary supplement formulation of claim 2, wherein the breed specific component includes one or more additives selected from the group consisting of biotin, zinc and DHA.

20. The dietary supplement formulation of claim 2, wherein the breed specific component includes one or more additives selected from the group consisting of vitamin C, sodium hexametaphosphate or some other phosphate group, Fucans (brown algae), natural abrasive, malic acid, zinc citrate and \acute{α}-lipoic acid.

21. The dietary supplement formulation of claim 2, wherein the breed specific component includes a combination of additives selected from the group consisting of:
(a) DHA, egg shell membrane, taurine and carnitine;
(b) eicosapentaenoic; chondroitin, hyaluronic acid, methylsulfonylmethane, glucosamine, esterified fatty acid complex, Chinese Skullcap, Black Catechu, creatine and ubidiquinone;
(c) DHA, carnitine taurine, vitamin A, vitamin E, vitamin B₂ and taurine;
(d) eicosapentaenoic acid; creatine, ubidiquinone, polyphenols, bioflavinoids, bilberry and carotenoids;
(e) DHA, taurine, antioxidants, vitamin E, vitamin C, vitamin A, and B vitamins;
(f) DHA; carnitine, taurine, vitamin A, vitamin E, vitamin B₂, taurine, egg shell membrane and glucosamine; and
(g) medium chain triglycerides, lecithin, and phospholipids, creatine, ubidiquinone, polyphenols, bioflavinoids, bilberry, carotenoids, chondroitin, hyaluronic acid, methylsulfonylmethane, glucosamine, esterified fatty acid complex, Chinese Skullcap and Black Catechu.

22. The dietary supplement formulation of claim 2, wherein the breed specific component includes lysine and taurine.

23. The dietary supplement formulation of claim 22, wherein the breed specific component also includes one or more additives selected from the group consisting of egg shell membrane, chondroitin, hyaluronic acid, methylsulfonylmethane, glucosamine and esterified fatty acid complex, lysine, antioxidants and cognitive support ingredients.

24. The dietary supplement formulation of claim 1, wherein said supplement is in a form selected from the group consisting of powder or meal, granular, pelleted and crumbled.
